(19) United States
(12) Patent Application Publication
(10) Pub. No.: US 2023/0298734 A1
(43) Pub. Date: Sep. 21, 2023

(54) COMPUTER SYSTEM FOR INTELLIGENT STRESS PREDICTION AND MANAGEMENT BASED ON SMARTPHONE DATA, AND METHOD OF THE SAME

(71) Applicant: Korea Advanced Institute of Science and Technology, Daejeon (KR)

(72) Inventors: Hwajung HONG, Daejeon (KR); Tae Wan KIM, Daejeon (KR)

(21) Appl. No.: 18/101,359

(22) Filed: Jan. 25, 2023

(30) Foreign Application Priority Data
Mar. 18, 2022 (KR) 10-2022-0033731

(51) Int. Cl. G16H 20/70; A61B 5/16; A61B 5/00

(52) U.S. Cl. CPC G16H 20/70; A61B 5/165; A61B 5/742

(57) ABSTRACT

Disclosed is a computer system and method for intelligent stress prediction and management based on smartphone data that may be configured to build a personalized stress prediction model for a user based on self-reported stress data input from the user during a preset period; and to understand and manage a stress of the user using the personalized stress prediction model. To understand and manage the stress of the user, the stress of the user may be predicted using the personalized stress prediction model, a stress intervention plan for helping the user to relieve the stress of the user may be set and provided, and a stress level of the user for each date may be provided in a form of a calendar.

### CROSS-REFERENCE TO RELATED APPLICATION

This application claims the priority benefit of Korean Patent Application No. 10-2022-0033731, filed on Mar. 18, 2022, in the Korean Intellectual Property Office, the disclosure of which is incorporated herein by reference in its entirety.

### BACKGROUND

### 1. Field of the Invention

The following description of various example embodiments relates to a computer system and method for understanding and managing a stress level of an individual user using a machine learning model during daily life.

This work was supported by the Institute of Information & communications Technology Planning & Evaluation (IITP) grant funded by the Korean government (MSIT) (No. 2022-0-00064, Development of Human Digital Twin Technologies for Prediction and Management of Emotion Workers' Mental Health Risks).

### 2. Description of the Related Art

Stress is an important factor that determines the quality of life and is an important task to be solved to improve the quality of life in Korean society. In terms of stress management, it is very important to correctly recognize and mediate one's own stress level. For example, although a level of stress perceived by a person is low, a level of stress actually felt by a body may be severe. In this case, the person may develop diseases caused by stress without correctly recognizing a stress level. The number of such cases is increasing.

In terms of stress measurement, stress measurement methods currently used in clinical practice include a self-diagnosis, a biosignal analysis, and a hormone analysis. The self-diagnosis relates to measuring a stress index through a test strip that includes items related to physical, behavioral, and psychological states for the recent one month and it has been reported that the examination result is greatly affected by a psychological state and a usual personality of a subject at the time of measurement. In contrast, an examination through the biosignal analysis relates to measuring a response state of a body to a tension of the body or a stressing situation by analyzing a heart rate variability, a skin conductance, and an electromyogram signal and is relatively objective. However, it has been reported that since the examination is performed in a subjective environment, it is difficult to determine an accurate physical stress level to a stressor due to a significant psychological factor. To solve such issues, it is emphasized that an unrestrained and non-aware individual stress measurement and management technique is required as a periodical and constant monitoring method in daily life.

Although a system for measuring a stress level of an individual based on sensor data through a wearable device and a smartphone application is proposed, the system is limited to a function of simply visualizing the stress level of the individual. However, a level of stress perceived by an individual is very subjective and is affected by numerous factors. Therefore, data collected by a smartphone and a wearable sensor may be affected by external factors. For example, the system has limitations in predicting stress caused by external events or environments uncollectable by a wearable device or a smartphone. In addition, users tend to rely more on a stress level prediction using technology than stress levels actually perceived by the users. This may reduce an individual's efforts to understand a stress and its causes by a corresponding user himself or herself. This phenomenon may prevent the user from correctly recognizing a stress level of the user.

Overall, design of a stress understanding and management system in which an individual-lead or subjective evaluation is integrated may be an important factor to be considered in a data-based and machine learning-based daily stress measurement system.

### SUMMARY

Various example embodiments provide a computer system and method for intelligent stress prediction and management based on smartphone data.

A method of a computer system according to various example embodiments may include building a personalized stress prediction model for a user based on self-reported stress data input from the user during a preset period; and understanding and managing a stress of the user using the personalized stress prediction model.

A computer system according to various example embodiments may include a memory; and a processor configured to connect to the memory and to execute at least one instruction stored in the memory. The processor may be configured to build a personalized stress prediction model for a user based on self-reported stress data input from the user during a preset period, and to understand and manage a stress of the user using the personalized stress prediction model.

Various example embodiments provide integrated technology for understanding and managing a personal stress using stress prediction technology and enable a user to self-manage a stress of the user. Here, through an explanation function for a stress level predicted for the user, the user may understand a cause of stress. This may improve reliability for technology according to various example embodiments and may assist the active use of this technology. Understanding of stress through a notification provided at a preset time interval during the day may improve a level of awareness for a stress level of the user and may help the user to correctly recognize the stress level of the user.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 is a diagram illustrating a configuration of a computer system according to various example embodiments;

FIG. 2 is a flowchart illustrating a method of a computer system according to various example embodiments;

FIG. 3 is a flowchart illustrating an operation of building a personalized stress prediction model of FIG. 2;

FIGS. 4A, 4B, and 4C illustrate an example of an operation of building a personalized stress prediction model of FIG. 2;

FIG. 5 is a flowchart illustrating a stress prediction operation in an operation of understanding and managing a stress of FIG. 2;

FIGS. 6A, 6B, 6C, 6D, and 6E illustrate an example of a stress prediction operation in an operation of understanding and managing a stress of FIG. 2;

FIGS. 7A and 7B illustrate an example of a stress intervention plan setting and providing operation in an operation of understanding and managing a stress of FIG. 2; and

FIGS. 8A and 8B illustrate an example of a stress verification operation in an operation of understanding and managing a stress of FIG. 2.

### DETAILED DESCRIPTION

Hereinafter, example embodiments will be described with reference to the accompanying drawings.

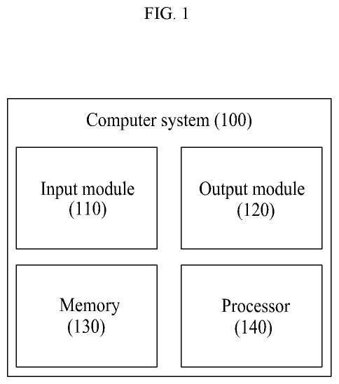

FIG. 1 is a diagram illustrating a configuration of a computer system 100 according to various example embodiments.

Referring to FIG. 1, the various example embodiments provide the computer system 100 for intelligent stress prediction and management based on smartphone data. The computer system 100 may include at least one of an input module 110, an output module 120, a memory 130, and a processor 140. In some example embodiments, at least one of components of the computer system 100 may be omitted and at least one another component may be added. In some example embodiments, at least two among the components of the computer system 100 may be implemented as a single integrated circuitry. Here, the components of the computer system 100 may be implemented as a single device (e.g., a smartphone of a user) and may be dividedly implemented with at least two individual devices (e.g., a server and a smartphone of a user).

The input module 110 may input a signal to be used for at least one component of the computer system 100. Here, the input module 110 may include at least one of an input device configured to directly input a signal to the computer system 100, a sensor device configured to detect an ambient change and to generate a signal, and a reception device configured to receive a signal from an external device. For example, the input device may include at least one of a camera module, a microphone, a mouse, and a keyboard. In some example embodiments, the sensor device may include at least one of a touch circuitry configured to detect a touch and a sensor circuitry configured to measure strength of force generated by the touch.

The output module 120 may output information to an outside of the computer system 100. The output module 120 may include at least one of a display device configured to visually output information, an audio output device configured to output information as an audio signal, and a transmission device configured to wirelessly transmit information. For example, the display device may include at least one of a display, a hologram device, and a projector. For example, the display device may be configured as a touchscreen through assembly to at least one of the touch circuitry and the sensor circuitry of the input module 110. For example, the audio output device may include at least one of a speaker and a receiver.

According to some example embodiments, the reception device and the transmission device may be implemented as a communication module. The communication module may communicate with the external device in the computer system 100. The communication module may establish a communication channel between the computer system 100 and the external device and may communicate with the external device through the communication channel. Here, the external device may include at least one of a satellite, a base station, a server, and another computer system. The communication module may include at least one of a wired communication module and a wireless communication module. The wired communication module may be connected to the external device in a wired manner and may communicate with the external device in the wired manner. The wireless communication module may include at least one of a near field communication module and a far field communication module. The near field communication module may communicate with the external device using a near field communication scheme. For example, the near field communication scheme may include at least one of Bluetooth, wireless fidelity (WiFi) direct, and infrared data association (IrDA). The far field communication module may communicate with the external device using a far field communication scheme. Here, the far field communication module may communicate with the external device over a network. For example, the network may include at least one of a cellular network, the Internet, and a computer network such as a local area network (LAN) and a wide area network (WAN).

The memory 130 may store a variety of data used by at least one component of the computer system 100. For example, the memory 130 may include at least one of a volatile memory and a non-volatile memory. Data may include at least one program and input data or output data related thereto. The program may be stored in the memory 130 as software including at least one instruction and may include at least one of an operating system (OS), middleware, and an application.

The processor 140 may control at least one component of the computer system 100 by executing the program of the memory 130. Through this, the processor 140 may perform data processing or operation. Here, the processor 140 may execute an instruction stored in the memory 130.

According to various example embodiments, the computer system 100 may assist individual-initiated stress understanding and management using a “user evaluation related to a predicted stress level” and an “explanation function.” Here, the “user evaluation related to the predicted stress level” represents that a user directly inputs an actual stress level of the user based on a stress level predicted by a machine learning algorithm. The “explanation function” refers to providing an explanation regarding how the machine learning algorithm predicts a stress and the user may verify data used for the machine learning algorithm to derive a prediction value.

The processor 140 may continuously collect smartphone usage data, that is, smartphone usage and sensor data and self-reported data related to a stress for a preset period of time at the beginning of usage (e.g., 10 to 15 days) using an application installed on a smartphone of the user. The processor 140 may build a personalized stress prediction model using the collected smartphone data and self-reported data.

The processor 140 may provide the user with stress prediction information from a prediction time to at a previous specific time interval, that is, at a specific time of the day using the personalized stress prediction model. The stress prediction information may include a predicted stress level and a period corresponding to the predicted stress level. For example, the predicted stress level may be expressed using one of three levels, that is, low, high, and very high. Through this, the user may perform a self-evaluation on an actual stress level perceived by the user based on the stress prediction information. The processor 140 may provide a stress analysis report by referring to the actual stress level of the user. The stress analysis report may include a final stress level, a period corresponding to the final stress level, and an explanation function related to data with a major influence on the final stress level. Here, the final stress level may be determined based on the input actual stress level. For example, the final stress level may be expressed as one of three levels, that is, low, high, and very high. The explanation function may represent providing an explanation regarding a method of predicting, by the personalized stress prediction model, the final stress level. That is, the processor 140 may provide the explanation function by visualizing a type of data or an aspect of data with a major influence on predicting the final stress level.

When the final stress level of the stress analysis report is high or higher, the processor 140 may determine an appropriate intervention point in time during an intervening time before providing subsequent stress prediction information and may provide a notification such that a stress intervention plan may be performed. The stress intervention plan refers to a simple activity that may be easily performed in daily life and may be generated, added, modified, and deleted by the user based on a taste or interest of the user.

The processor 140 may perform a stress verification operation. Here, the processor 140 may provide the user with a stress level for each period. Through this, the user may verify a stress level of a desired period and may verify a stress trend. In detail, the processor 140 may provide the user with a stress level for each date using a user interface in a form of a calendar.

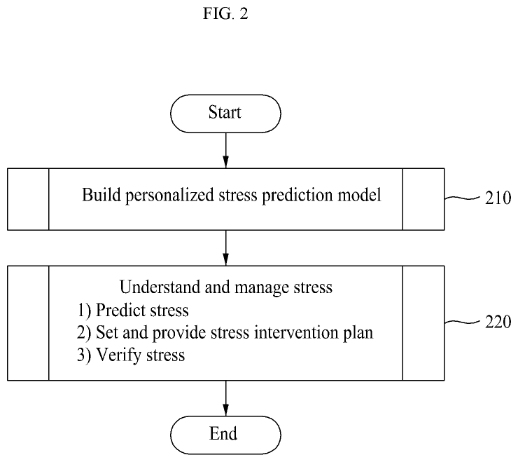

FIG. 2 is a flowchart illustrating a method of the computer system 100 according to various example embodiments.

Referring to FIG. 2, in operation 210, the computer system 100 may build a personalized stress prediction model. Here, the processor 140 may build the personalized stress prediction model through a self-reported stress level input process of the user. Operation 210 may be performed for a preset period of time in an initial stage, for example, for 10 to 15 days.

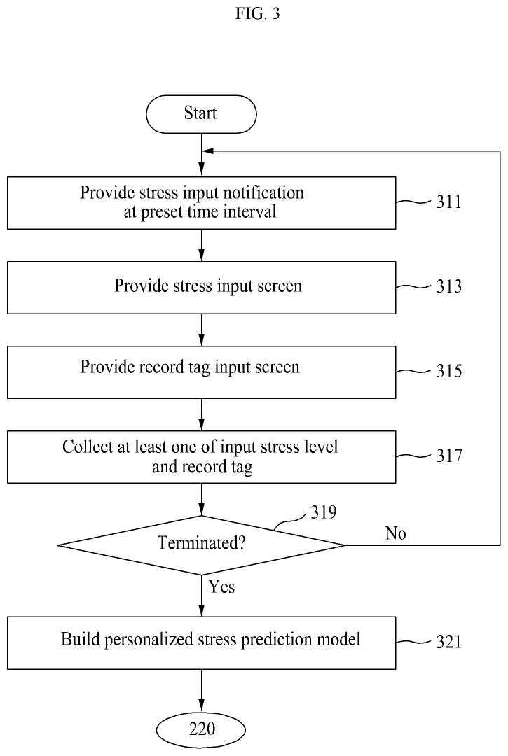

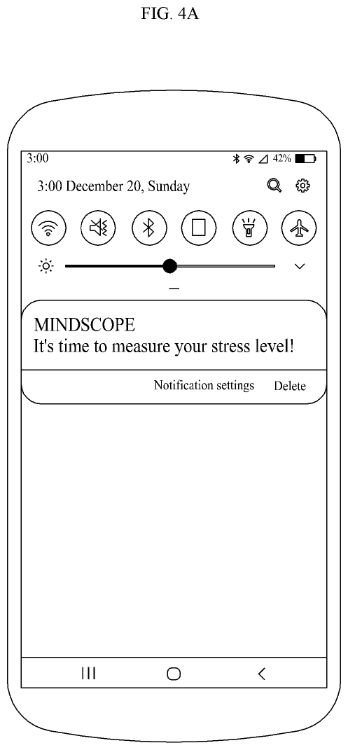

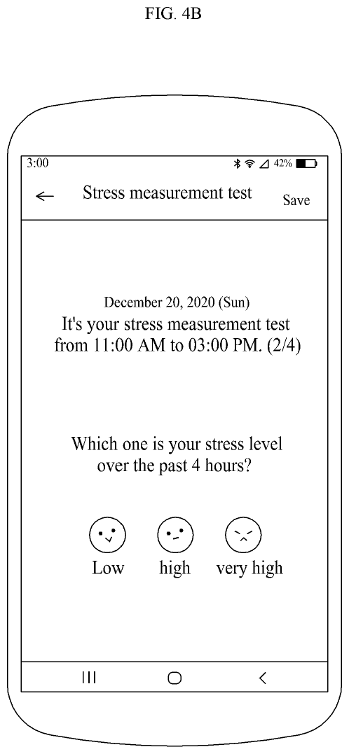

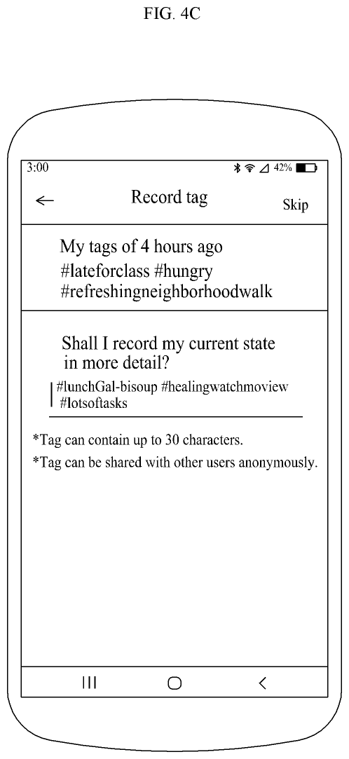

In detail, the processor 140 may collect self-reported data required for the user to build the stress prediction model at a preset time interval. To this end, a smartphone notification function and a user interface (e.g., a graphical user interface (GUI)) of an application installed on a smartphone of the user may be used. The user may input a stress level of the user at a preset time interval. In some example embodiments, the processor 140 may collect smartphone usage data, that is, smartphone usage and sensor data with self-reported data. Therefore, the processor 140 may build the personalized stress prediction model for the user through learning of the machine learning algorithm based on the collected self-reported data. In some example embodiments, the processor 140 may build the personalized stress prediction model based on the collected self-reported data and the collected smartphone usage data. Further description related thereto will be made with reference to FIG. 3 and FIGS. 4A, 4B, and 4C.

FIG. 3 is a flowchart illustrating operation 210 of building the personalized stress prediction model of FIG. 2. FIGS. 4A, 4B, and 4C illustrate an example of operation 210 of building the personalized stress prediction model of FIG. 2.

Referring to FIG. 3, in operation 311, the computer system 100 may provide a stress input notification to the user at a preset time interval. For example, referring to FIG. 4A, the processor 140 may provide the stress input notification through a user interface.

In operation 313, the computer system 100 may provide a stress input screen. For example, referring to FIG. 4B, the processor 140 may provide the stress input screen through a user interface. Through this, the user may input a stress level of the user through the stress input screen. Here, items representing different stress levels, respectively, may be displayed on the stress input screen and the user may select a single item from among the items based on a stress level of the user.

In operation 315, the computer system 100 may provide a record tag input screen. Referring to FIG. 4C, the processor 140 may provide the record tag input screen through a user interface. Through this, the user may input a record tag for a state of the user through the record tag input screen. Here, although the record tag input screen is provided, the user may not input the record tag.

In operation 317, the computer system 100 may collect at least one of the stress level and the record tag input from the user as self-reported data. Since at least one of the stress level and the record tag input through the user interface is submitted, the processor 140 may collect the self-reported data. Here, a current status of participation into inputting the stress level may be verified.

In operation 319, the computer system 100 may determine whether to terminate collecting of the self-reported data. The processor 140 may determine whether a preset period of time in an initial stage has elapsed. When the preset period of time elapses, the processor 140 may determine that collecting of the self-reported data may be terminated. When the preset period of time does not elapse, the processor 140 may determine that collecting of the self-reported data may not be terminated and may repeatedly perform operations 311 to 317 by returning to operation 311.

When it is determined that collecting of the self-reported data may be terminated in operation 319, the computer system 100 may build the personalized stress prediction model for the user in operation 321. The processor 140 may build the personalized stress prediction model through learning of the machine learning algorithm based on self-reported data collected during a preset period of time. By returning to FIG. 2, the computer system 100 may perform operation 220.

Referring again to FIG. 2, in operation 220, the computer system 100 may understand and manage a stress using the personalized stress prediction model. When the personalized stress prediction model is built, the computer system 100 may continuously use the personalized stress prediction model. In operation 220, the computer system 100 may perform the following three operations, for example, a stress prediction operation, a stress intervention plan setting and providing operation, and a stress verification operation. The operations may be performed according to an intent of the user regardless of order.

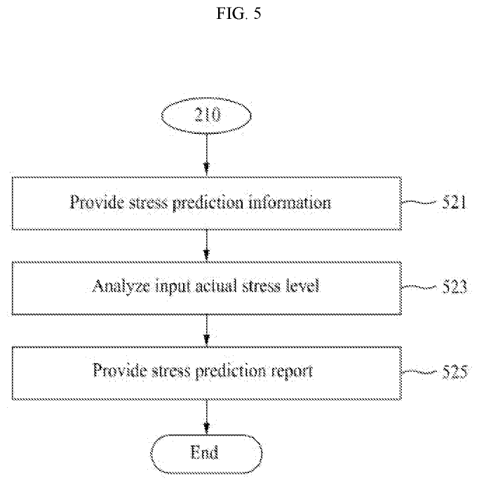

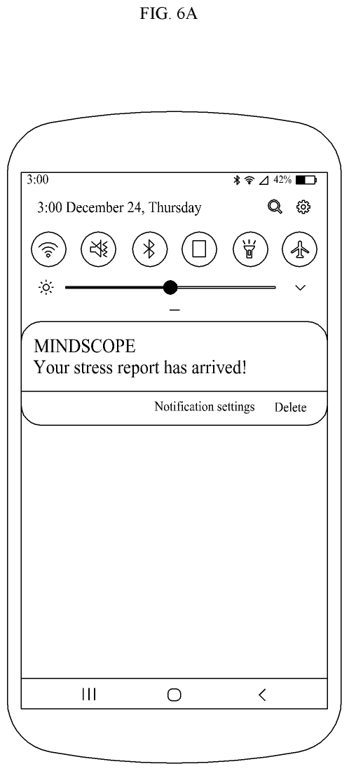

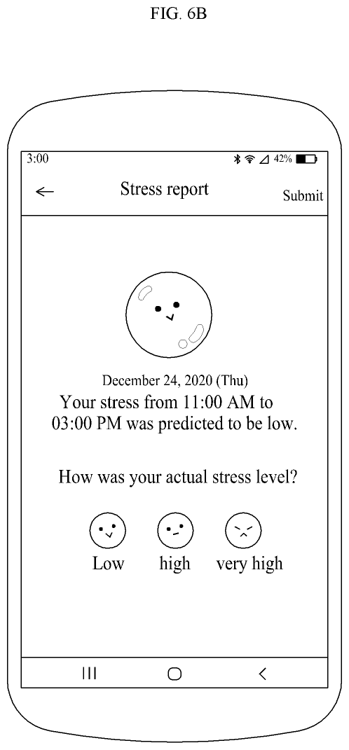

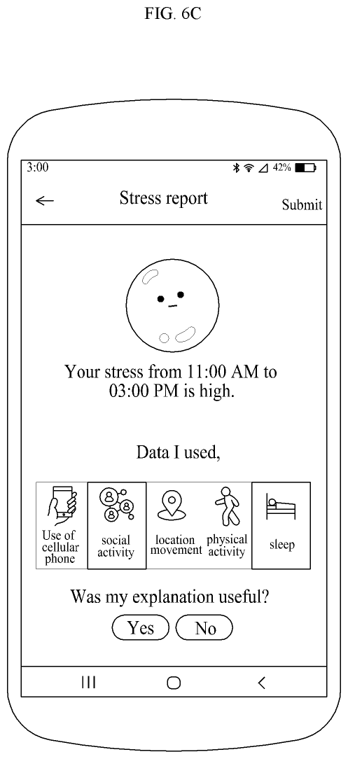

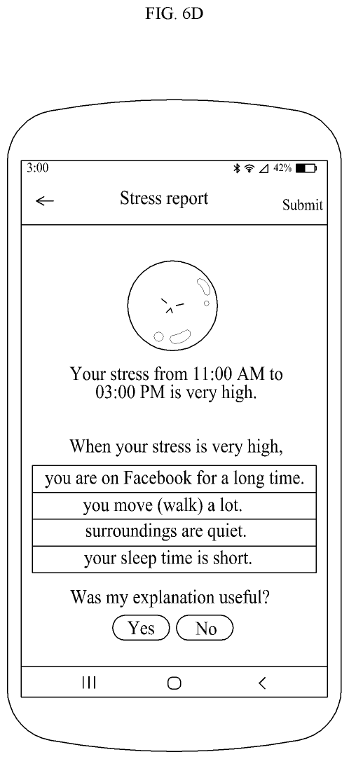

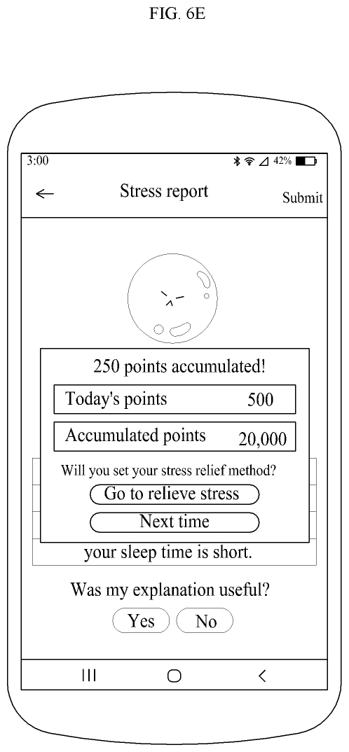

In detail, in operation 220, the processor 140 may perform the stress prediction operation using the personalized stress prediction model. Here, the processor 140 may predict a stress of the user with respect to a previous specific time interval. Further description related thereto will be made with reference to FIG. 5 and FIGS. 6A, 6B, 6C, 6D, and 6E.

FIG. 5 is a flowchart illustrating a stress prediction operation in operation 220 of understanding and managing the stress of FIG. 2. FIGS. 6A, 6B, 6C, 6D, and 6E illustrate an example of the stress prediction operation in operation 220 of understanding and managing the stress of FIG. 2.

Referring to FIG. 5, in operation 521, the computer system 100 may provide the user with stress prediction information of a previous specific time interval using the personalized stress prediction model. Here, the processor 140 may provide the user with stress prediction information of the previous specific time interval using a user interface with a graphical function. For example, referring to FIG. 6A, the processor 140 may provide a notification for stress prediction information through a user interface. Referring to FIG. 6B, the processor 140 may provide stress prediction information through a user interface. The stress prediction information may include a predicted stress level and a period corresponding to the predicted stress level. For example, the predicted stress level may be expressed as one of three levels, that is, low, high, and very high. Here, the period corresponding to the predicted stress level may be expressed as a period, for example, from 11:00 AM to 3:00 PM on Dec. 24, 2020. In addition, the processor 140 may request the user to input an actual stress level while providing the stress prediction information. Through this, the user may input the actual stress level of the user through the user interface. For example, referring to FIG. 6B, the processor 140 may display items that represent different stress levels, respectively, through the user interface and the user may select a single item from among the items according to the actual stress level.

In operation 523, the computer system 100 may analyze the input actual stress level using the personalized stress prediction model. To this end, the input actual stress level may be fed back to the personalized stress prediction model.

In operation 525, the computer system 100 may provide a stress analysis report to the user using the personalized stress prediction model. The stress analysis report may include a final stress level, a period corresponding to the final stress level, and an explanation function related to data with a major influence on the final stress level. Here, the final stress level may be determined based on the input actual stress level. For example, the final stress level may be expressed as one of three levels, that is, low, high, and very high. Here, the period corresponding to the final stress level may be expressed as a period, for example, from 11:00 AM to 3:00 PM on Dec. 24, 2020. The explanation function may represent providing an explanation regarding a method of predicting, by the personalized stress prediction model, the final stress level. That is, the processor 140 may provide the explanation function by visualizing a type of data or an aspect of data with a major influence on predicting the final stress level.

Here, the explanation function may be implemented using the following explanation methods, for example, a first explanation method and a second explanation method. According to the first explanation method, the processor 140 may provide the explanation function as shown in FIG. 6C. In detail, the processor 140 may visualize a category of data with a major influence on predicting the final stress level. Here, for data to be visualized, five categories may be configured as follows: use of a cellular phone, a social activity, a location movement, a physical activity, and sleep. Here, the processor 140 may display items that represent the categories, respectively, and may emphasize a category of data with a major influence on predicting the final stress level. According to the second explanation method, the processor 140 may provide the explanation function as shown in FIG. 6D. In detail, the processor 140 may visualize an aspect of sensor data and user activity with a major influence on predicting the final stress level. Here, the processor 140 may express the aspect of sensor data and the user activity with a major influence on predicting the final stress level as one or more sentences (e.g., when your stress level is high, you are on Facebook for a long time; move (walk) a lot; surroundings are quiet; and your sleep time is short). In addition, while providing the explanation function, the processor 140 may collect user feedback by providing a question asking about usefulness of the explanation function through a user interface.

In some example embodiments, referring to FIG. 6E, when verification of the stress analysis report is verified, the computer system 100 may provide the user with a reward notification through points. On a screen for the reward notification, the following information and buttons may be displayed: 1) points accumulated due to an input of an actual stress level, 2) points accumulated during the day, 3) total accumulated points, and 4) screen navigation buttons for implementing a stress intervention plan.

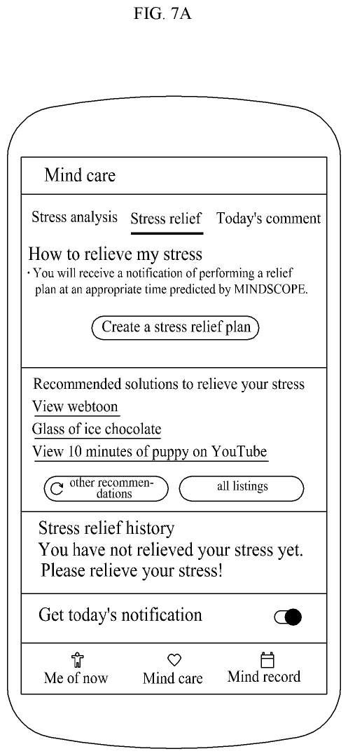

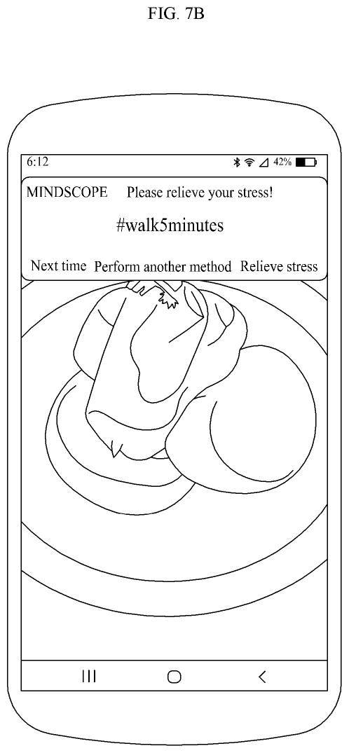

In operation 220, the processor 140 may perform a stress intervention plan setting and providing operation. Here, the processor 140 may set a stress intervention plan for the user. The stress intervention plan may represent at least one activity that may help the user to relieve a stress of the user. The processor 140 may provide the stress intervention plan to the user. Here, the processor 140 may recommend at least one activity to the user to help the user to relieve the stress. Further description related thereto will be made with reference to FIGS. 7A and 7B.

FIGS. 7A and 7B illustrate an example of a stress intervention plan setting and providing operation in operation 220 of understanding and managing the stress of FIG. 2.

In detail, the processor 140 may set a stress intervention plan for the user. For example, referring to FIG. 7A, the processor 140 may provide a stress intervention plan setting screen through a user interface. Through this, the processor 140 may set the stress intervention plan for the user, each activity, and a time for performing a corresponding activity based on data input from the user. Here, the user may input the data based on a taste and interest of the user. Here, each activity as the stress intervention plan may be newly generated and may also be added, modified, and deleted.

The processor 140 may provide the stress intervention plan to the user. Here, the processor 140 may recommend at least one activity to the user to help the user to relieve the stress. Here, when a plurality of activities is set to the stress intervention plan, the processor 140 may suggest at least a portion of the set activities. The processor 140 may provide the stress intervention plan to the user based on a recent final stress level. For example, referring to FIG. 7B, when the recent final stress level is high or higher, the processor 140 may provide a notification such that the stress intervention plan may be performed through a user interface. The processor 140 may visualize an activity as the stress intervention plan, a time for performing the corresponding activity, and at least one button. The at least one button may include a button for postponing the suggested activity, a button for receiving recommendation on another activity different from the suggested activity, and a button for inputting a completion of the suggested activity. The user may input a completion of the stress intervention plan using a stress relief button.

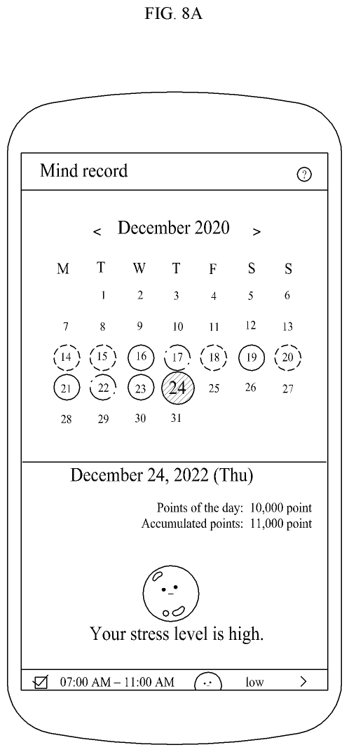

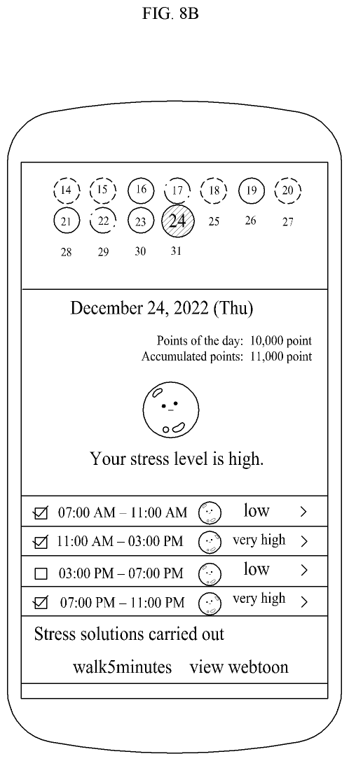

In operation 220, the processor 140 may perform a stress verification operation. Here, the processor 140 may provide a stress level for each period. Through this, the user may verify a stress level of a desired period and may verify a stress trend. Further description related thereto will be made with reference to FIGS. 8A and 8B.

FIGS. 8A and 8B illustrate an example of a stress verification operation in operation 220 of understanding and managing the stress of FIG. 2.

In detail, the processor 140 may provide a stress level for each date to the user using a user interface in a form of a calendar. For example, referring to FIG. 8A, the processor 140 may express an average stress level of a corresponding day on the calendar. In this manner, the user may verify a trend and flow of stress for one month. Referring to FIG. 8B, in response to a selection on a specific date from the user, the processor 140 may provide a stress report of a corresponding date to the user. The stress report may include an average stress level of a corresponding date, points accumulated and total points on the corresponding date, final stress levels in stress analysis reports provided on the corresponding date, and whether an actual stress level is input from the user. When the actual stress level is not input from the user, a predicted stress level in stress prediction information may be displayed instead of a final stress level and may be used to calculate the average stress level of the corresponding date.

Various example embodiments provide integrated technology for understanding and managing a personal stress using stress prediction technology and enables a user to self-manage a stress of the user. Here, through an explanation function for a stress level predicted for the user, the user may understand a cause of the stress. This may improve reliability for technology according to various example embodiments and may assist the active use of this technology. Understanding of stress through a notification provided at a preset time interval during the day may improve a level of awareness for a stress level of the user and may help the user to correctly recognize the stress level of the user.

For example, various example embodiments provide the computer system 100 and a method for intelligent stress prediction and management based on smartphone data.

According to various example embodiments, the method of the computer system 100 may include operation 210 of building a personalized stress prediction model for a user based on self-reported stress data input from the user during a preset period, and operation 220 of understanding and managing a stress of the user using the personalized stress prediction model.

According to various example embodiments, operation 220 of understanding and managing the stress of the user may include predicting the stress of the user using the personalized stress prediction model.

According to various example embodiments, the predicting of the stress of the user may include operation 521 of providing stress prediction information that includes a predicted stress level for a preset time interval through the personalized stress prediction model, operation 523 of analyzing an input actual stress level of the user based on the predicted stress level, using the personalized stress prediction model, and operation 525 of providing a stress analysis report that includes a final stress level predicted from the actual stress level through the personalized stress prediction model.

According to various example embodiments, the stress analysis report may include an explanation function of describing data with an influence on the personalized stress prediction model in predicting the final stress level. Here, the data may be determined from smartphone usage data of the user.

According to various example embodiments, operation 210 of building the personalized stress prediction model may include collecting smartphone usage data of the user with the self-reported stress data and building the personalized stress prediction model based on the self-reported stress data and the smartphone usage data.

According to various example embodiments, operation 210 of building the personalized stress prediction model may include collecting the self-reported stress data during the preset period, and operation 321 of building the personalized stress prediction model based on the self-reported stress data

According to various example embodiments, the collecting of the self-reported stress data may include operation 311 of providing a stress input notification at a preset time interval, operation 313 of providing a stress input screen for the user to input a stress level of the user, and operation 317 of collecting the stress level of the user input through the stress input screen as the self-reported stress data.

According to various example embodiments, the collecting of the self-reported stress data may further include operation 315 of, in response to an input of the stress level of the user through the stress input screen, providing a record tag input screen for the user to input a record tag for a state of the user, and operation 317 of, in response to an input of the record tag through the record tag input screen, collecting the stress level of the user and the record tag as the self-reported stress data.

According to various example embodiments, operation 220 of understanding and managing the stress of the user may include at least one of setting a stress intervention plan for helping the user to relieve the stress of the user, and providing a preset stress intervention plan for helping the user to relieve the stress of the user.

According to various example embodiments, operation 220 of understanding and managing the stress of the user may include providing an average stress level for each date predicted through the personalized stress prediction model, in a form of a calendar.

According to various example embodiments, operation 220 of understanding and managing the stress of the user may further include displaying an average stress level on a selected date, whether an actual stress level is input from the user at a preset time interval on the selected date, a final stress level in a stress analysis report when the actual stress level is input, and a predicted stress level of stress prediction information when the actual stress level is not input.

According to various example embodiments, the computer system 100 may include the memory 130, and the processor 140 configured to connect to the memory 130 and to execute at least one instruction stored in the memory 130.

According to various example embodiments, the processor 140 may be configured to build a personalized stress prediction model for a user based on self-reported stress data input from the user during a preset period, and to understand and manage a stress of the user using the personalized stress prediction model.

According to various example embodiments, the processor 140 may be configured to predict the stress of the user using the personalized stress prediction model.

According to various example embodiments, the processor 140 may be configured to provide stress prediction information that includes a predicted stress level for a preset time interval through the personalized stress prediction model, to analyze an input actual stress level of the user based on the predicted stress level, using the personalized stress prediction model, and to provide a stress analysis report that includes a final stress level predicted from the actual stress level through the personalized stress prediction model.

According to various example embodiments, the stress analysis report may include an explanation function of describing data with an influence on the personalized stress prediction model in predicting the final stress level. Here, the data may be determined from smartphone usage data of the user

According to various example embodiments, the processor 140 may be configured to collect smartphone usage data of the user with the self-reported stress data and to build the personalized stress prediction model based on the self-reported stress data and the smartphone usage data.

According to various example embodiments, the processor 140 may be configured to provide a stress input notification at a preset time interval, to provide a stress input screen for the user to input a stress level of the user, and to collect the stress level of the user input through the stress input screen as the self-reported stress data.

According to various example embodiments, the processor 140 may be further configured to, in response to an input of the stress level of the user through the stress input screen, provide a record tag input screen for the user to input a record tag for a state of the user, and in response to an input of the record tag through the record tag input screen, collect the stress level of the user and the record tag as the self-reported stress data.

According to various example embodiments, the processor 140 may be configured to set a stress intervention plan for helping the user to relieve the stress of the user, and to provide a preset stress intervention plan for helping the user to relieve the stress of the user.

According to various example embodiments, the processor 140 may be configured to provide a stress level for each date predicted through the personalized stress prediction model, in a form of a calendar and to display an average stress level of a selected date, whether an actual stress level is input from the user at a preset time interval on the selected date, a final stress level in a stress analysis report when the actual stress level is input, and a predicted stress level of stress prediction information when the actual stress level is not input.

The apparatuses described herein may be implemented using hardware components, software components, and/or a combination of the hardware components and the software components. For example, a processing device and components described herein may be implemented using one or more general-purpose or special purpose computers, for example, a processor, a controller, an arithmetic logic unit (ALU), a digital signal processor, a microcomputer, a field programmable gate array (FPGA), a programmable logic unit (PLU), a microprocessor, or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will be appreciated that the processing device may include multiple processing elements and/or multiple types of processing elements. For example, the processing device may include multiple processors or a processor and a controller. In addition, other processing configurations are possible, such as parallel processors.

The software may include a computer program, a piece of code, an instruction, or some combinations thereof, for independently or collectively instructing or configuring the processing device to operate as desired. Software and/or data may be embodied in any type of machine, component, physical equipment, computer storage medium or device, to provide instructions or data to the processing device or be interpreted by the processing device. The software also may be distributed over network coupled computer systems so that the software is stored and executed in a distributed fashion. The software and data may be stored by one or more computer readable storage mediums.

The methods according to various example embodiments may be implemented in a form of a program instruction executable through various computer methods and recorded in computer-readable media. Here, the media may be to continuously store a computer-executable program or to temporarily store the same for execution or download. The media may be various types of recording methods or storage methods in which single hardware or a plurality of hardware is combined and may be distributed over a network without being limited to a medium that is directly connected to a computer system. Examples of the media include magnetic media such as hard disks, floppy disks, and magnetic tapes; optical media such as CD ROM and DVD; magneto-optical media such as floptical disks; and hardware devices that are specially configured to store and perform program instructions, such as read-only memory (ROM), random access memory (RAM), flash memory, and the like. Examples of other media may include recording media and storage media managed by an app store that distributes applications or a site, a server, and the like that supplies and distributes other various types of software.

Various example embodiments and the terms used herein are not construed to limit description disclosed herein to a specific implementation and should be understood to include various modifications, equivalents, and/or substitutions of a corresponding example embodiment. In the drawings, like reference numerals refer to like components throughout the present specification. The singular forms “a,” “an,” and “the” are intended to include the plural forms as well, unless the context clearly indicates otherwise. Herein, the expressions, “A or B,” “at least one of A and/or B,” “A, B, or C,” “at least one of A, B, and/or C,” and the like may include any possible combinations of listed items. Terms “first,” “second,” etc., are used to describe corresponding components regardless of order or importance and the terms are simply used to distinguish one component from another component. The components should not be limited by the terms. When a component (e.g., a first component) is described to be “(functionally or communicatively) connected to” or “accessed to” another component (e.g., a second component), the component may be directly connected to the other component or may be connected through still another component (e.g., a third component).

The term “module” used herein may include a unit configured as hardware, software, or firmware, and may be interchangeably used with the terms, for example, “logic,” “logic block,” “part,” “circuit,” etc. The module may be an integrally configured part, a minimum unit that performs at least function, or a portion thereof. For example, the module may be configured as an application-specific integrated circuit (ASIC).

According to various example embodiments, each of the components (e.g., module or program) may include a singular object or a plurality of objects. According to various example embodiments, at least one of the components or operations may be omitted. Alternatively, at least one another component or operation may be added. Alternatively or additionally, a plurality of components (e.g., module or program) may be integrated into a single component. In this case, the integrated component may perform one or more functions of each of the components in the same or similar manner as it is performed by a corresponding component before integration. According to various example embodiments, operations performed by a module, a program, or another component may be performed in a sequential, parallel, iterative, or heuristic manner. Alternatively, at least one of the operations may be performed in different sequence or omitted. Alternatively, at least one another operation may be added.

The foregoing description has been provided for purposes of illustration and description. It is not intended to be exhaustive or to limit the disclosure. Individual elements or features of a particular example embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method of a computer system, comprising:
building a personalized stress prediction model for a user based on self-reported stress data input from the user during a preset period; and
understanding and managing a stress of the user using the personalized stress prediction model.

2. The method of claim 1, wherein the understanding and the managing of the stress of the user comprises predicting the stress of the user using the personalized stress prediction model.

3. The method of claim 2, wherein the predicting of the stress of the user comprises:
providing stress prediction information that has a predicted stress level for a preset time interval through the personalized stress prediction model;
analyzing an input actual stress level of the user based on the predicted stress level, using the personalized stress prediction model; and
providing a stress analysis report that has a final stress level predicted from the actual stress level through the personalized stress prediction model.

4. The method of claim 3, wherein the stress analysis report further has an explanation function of describing data with an influence on the personalized stress prediction model in predicting the final stress level.

5. The method of claim 4, wherein the building of the personalized stress prediction model comprises collecting smartphone usage data of the user with the self-reported stress data and building the personalized stress prediction model based on the self-reported stress data and the smartphone usage data, and
the data is determined from the smartphone usage data of the user.

6. The method of claim 1, wherein the building of the personalized stress prediction model comprises:
collecting the self-reported stress data during the preset period; and
building the personalized stress prediction model based on the self-reported stress data, and
the collecting of the self-reported stress data comprises:
providing a stress input notification at a preset time interval;
providing a stress input screen for the user to input a stress level of the user; and
collecting the stress level of the user input through the stress input screen as the self-reported stress data.

7. The method of claim 6, wherein the collecting of the self-reported stress data further comprises:
in response to an input of the stress level of the user through the stress input screen, providing a record tag input screen for the user to input a record tag for a state of the user; and
in response to an input of the record tag through the record tag input screen, collecting the stress level of the user and the record tag as the self-reported stress data.

8. The method of claim 1, wherein the understanding and the managing of the stress of the user comprises at least one of:
setting a stress intervention plan for helping the user to relieve the stress of the user; and
providing a preset stress intervention plan for helping the user to relieve the stress of the user.

9. The method of claim 1, wherein the understanding and the managing of the stress of the user comprises providing an average stress level for each date predicted through the personalized stress prediction model, in a form of a calendar.

10. The method of claim 9, wherein the understanding and the managing of the stress of the user further comprises displaying an average stress level on a selected date, whether an actual stress level is input from the user at a preset time interval on the selected date, a final stress level in a stress analysis report when the actual stress level is input, and a predicted stress level of stress prediction information when the actual stress level is not input.

11. A computer system comprising:
a memory; and
a processor configured to connect to the memory and to execute at least one instruction stored in the memory,
wherein the processor is configured to,
build a personalized stress prediction model for a user based on self-reported stress data input from the user during a preset period, and
understand and manage a stress of the user using the personalized stress prediction model.

12. The computer system of claim 11, wherein the processor is configured to predict the stress of the user using the personalized stress prediction model.

13. The computer system of claim 12, wherein the processor is configured to,
provide stress prediction information that has a predicted stress level for a preset time interval through the personalized stress prediction model,
analyze an input actual stress level of the user based on the predicted stress level, using the personalized stress prediction model, and
provide a stress analysis report that has a final stress level predicted from the actual stress level through the personalized stress prediction model.

14. The computer system of claim 13, wherein the stress analysis report has an explanation function of describing data with an influence on the personalized stress prediction model in predicting the final stress level.

15. The computer system of claim 14, wherein the processor is configured to collect smartphone usage data of the user with the self-reported stress data and to build the personalized stress prediction model based on the self-reported stress data and the smartphone usage data, and
the data is determined from the smartphone usage data of the user.

16. The computer system of claim 11, wherein the processor is configured to,
provide a stress input notification at a preset time interval,
provide a stress input screen for the user to input a stress level of the user, and
collect the stress level of the user input through the stress input screen as the self-reported stress data.

17. The computer system of claim 16, wherein the processor is further configured to,
in response to an input of the stress level of the user through the stress input screen, provide a record tag input screen for the user to input a record tag for a state of the user, and
in response to an input of the record tag through the record tag input screen, collect the stress level of the user and the record tag as the self-reported stress data.

18. The computer system of claim 11, wherein the processor is configured to,
set a stress intervention plan for helping the user to relieve the stress of the user, and
provide a preset stress intervention plan for helping the user to relieve the stress of the user.

19. The computer system of claim 11, wherein the processor is configured to,
provide an average stress level for each date predicted through the personalized stress prediction model, in a form of a calendar, and
display an average stress level on a selected date, whether an actual stress level is input from the user at a preset time interval on the selected date, a final stress level in a stress analysis report when the actual stress level is input, and a predicted stress level of stress prediction information when the actual stress level is not input.

20. A non-transitory computer-readable recording medium storing at least one program to implement a method for intelligent stress understanding and management on a computer system, wherein the method comprises:
building a personalized stress prediction model for a user based on self-reported stress data input from the user during a preset period; and
understanding and managing a stress of the user using the personalized stress prediction model.
